(19) United States
(12) Patent Application Publication
(10) Pub. No.: US 2011/0137189 A1
(43) Pub. Date: Jun. 9, 2011

(54) PHYSIOLOGICAL SIGNAL SENSING SYSTEM WITHOUT TIME AND PLACE CONTRAINT AND ITS METHOD

(75) Inventors: Bo-Jau KUO, Taipei City (TW); Fu-Jen KUO, Taipei City (TW); Cheng-Chun LEE, Taipei City (TW); Ching-Hsiu YANG, Taipei City (TW)

(73) Assignee: NATIONAL YANG-MING UNIVERSITY, Taipei City (TW)

(21) Appl. No.: 12/695,847

(22) Filed: Jan. 28, 2010

(30) Foreign Application Priority Data
Dec. 3, 2009 (TW) 098141299

(51) Int. Cl. A61B 5/0408

(52) U.S. Cl. 600/509

(57) ABSTRACT

A physiological signal sensing system and a physiological signal sensing method applied in any time and place are disclosed. The present invention is used by applying wireless transmitting electrocardiogram (ECG) data and contactless charging so as to achieve the purpose of sensing physiological signals without any time and place constraints.

### FIELD OF THE INVENTION

The present invention is related to a physiological signal system sensing method without time and place constraint and its method.

### BACKGROUND OF THE INVENTION

### 1. Development for Non-Invasive Diagnostic Techniques

Medical science has been advanced to be divided into a structural study and a functional study. In the functional study, physiologists have developed various procedures to measure functions for every organ and tissue of the body. Over hundreds of years, most functions for every organ have been measured and diagnosed by corresponding procedures. However, past development is mainly considered to discover and thus focus on accuracy in signal measurement. In order to achieve this purpose, many invasive tools and techniques would be applied. For example, cardiac catheterization is applied by stretching a tube through an artery to reach a heart. However, it is not only painful but also dangerous, so that it fails to care for subject's feelings. Nowadays, the invasive techniques have been developed to the ultimate and another concept has been formed gradually, i.e. non-invasive diagnostic techniques. By comparing the invasive techniques, the non-invasive techniques merely use a non-invasive process through applying painless tools and techniques to measure and diagnose the functions of the organs. Since the non-invasive techniques does not include invasive, they cannot obtain the most accurate physiological signals and thus includes no satisfactory accuracy and practicality. In recent years, the techniques for signal detecting and processing have been developed, especially in software engineering. Thus, the weakness for the non-invasive techniques could be overcome by computer to obtain valuable results. The Heart Rate Variability (HRV) analysis (Anonymous 1996) is a representative for the non-invasive diagnostic techniques. The HRV analysis is applied by electrode on the body surface to measure electrocardiogram (ECG) signals and quantitative indicators for autonomic nerves functions would be obtained by processing complicated digital signals. Based on this technique, some functions or diseases such as depth of anesthesia (Yang et al. 1996), brain death (Kuo et al. 1997), prognosis in severely ill patients (Yien et al. 1997), ageing (Kuo et al. 1999), and gender differences (Kuo et al. 1999) have been diagnosed. If it is considered for convenience and comfort of the subjects, the non-invasive technique still has much of development space. Animal experiment is actively used as various researches by these indicators (Kuo et al. 2005).

In addition, detection for three-axis acceleration is an important physical activity indicator, since it is measured without directly contacting the body. The measurements for detecting ECG signals and physical activity are two important physiological signals for designing sensor to increase more reliable and convenient for quantifying various change of physiological signals in clinical (health person and patient) and research (animal physiology and behavior).

### 2. Importance for Wireless Physiological Signals Monitoring and Collecting System of to Medicine

The conventional long-time physiological signals detecting system is configured on the traditional wire transmission technology. The subjects must be pasted on their body with many electrodes, and these electrodes would be connected to an amplifier through conducting wires to perform analog-digital converting and process digital signals. There are many wiring on the subjects' body, so that it is difficult to move for the subjects and is hard to be applied in life warning. Accordingly, it is quite inconvenient. Furthermore, it is difficult for many measuring methods and merely few professional technical persons with much training could perform all detecting methods of physiological phenomenon. Thus, it is not easy to use. A wireless system has been developed by some factories lately, but most wireless systems do not escape wire constraint. Most system electrode is still connected to the host through the conducting wires. After the amplifying and analog to digital conversion system, the digital signals will be wireless transmitted by the micro-controller. It would be convenient for the user; even so, these conducting wires would form certain constraint for the users. In addition, the conducting wire is a noise source. It is not easy to carry since the most instruments are oversized.

Now, most human recorder includes a weakness, i.e. fails to monitor the patient's health status in time, and the real-time record in the early warning function may be lost. Therefore, it is difficult for any warning.

However, the most common behavior monitoring in experimental animals or general animals is implemented by non-invasive video recording. Thus, it lacks for some physiological signals observations. The research for physiological signals monitoring is implemented by applying conducting wireless to be connected to a signal amplifier host, whereby the animal's behavior is limited, and the problem merely is based on the technologic limit. If the animal's physiology and behavior are measured under wireless freedom status, the signal should be more correct detected.

### 3. Importance for Monitoring Ultra-Long Time Heart Rate and Autonomic Nervous Activity and the Activity

In medicine, there are many diseases take a long-time observation to understand the possible causes. It is important for long-time monitoring in patient care and it is more important for long-time tracking therapy. Further, there are many applications for the heart rate and HRV techniques in clinical. For example, Wolf et al. (1978) provided the mortality of myocardial infraction patient would be increased when HRV is decreased. Myers et al (1988) found that the cardiac patient with higher high-frequency component (HF) is less susceptible to sudden death. Binkley et al. (1991) also found that congestive heart failure patients have lower HF and higher LF/HF. Langewitz et al. (1994) found that hypertensive patients have lower HF and higher low-frequency (LF) according to the research of spectrum analysis for 34 hypertensive patients and 41 borderline hypertensive

patients. Singh et al (1998) found that hypertensive patients have lower HRV and normal blood-pressure men with lower HRV easily result in essential hypertension by researching the Framingham heart study with spectrum analysis to trace 24 hrs electrocardiogram recordings of ethnic groups, especially in LF as a best predictive indicator. That is to say, HRV spectrum analysis is not only used for measuring autonomic nervous activity, but also used as tool for diagnosing and forecasting fatal disease to occur. In addition, higher HF measurement represents higher vagus nerve activity to protect cardiovascular system and decrease the mortality. Therefore, it can provide real patients with time and the long-term care if long-time tracing these information and conditions.

The activity calculated by triaxial accelerator linear and immediately responds physical activity level of the body, so that its uses will be wide. It is found that the parameter has a particular change in responding long-time activity. Through using quantification of long-time activity, condition, health condition and lifestyle can been provided, in which it is more important for assessing awake by this parameter. Further, the amount of long-time total activity is another reliable physiological index.

### 4. Importance for Monitoring Ultra-Long Time Animal Physiology and Behavior

Observation of animal behavior often needs accurate physical values to provide free moving (be closer to natural conditions) for animals, so that implanting electrodes is the most common process. However, various long time physiological changes (heart rate, cardiac autonomic nerve function, activity and behavior, etc.) so far are difficult to have complete information due to power limit, especially in the evolution of physiological signals with age changes as complete life observation. This endless power supply will be an important breakthrough in study of animal behavior.

### 5. Importance for Ultra-Long Time Radio Stimulation System

The electrical stimulation is a common treatment or research in the human or animal medical experiments. However, this method often includes some problems. For example, it is not consentient for the subjects because of wired, so that the medical treatment or research cannot be long time provided and the subject may feel the pressure. Therefore, the future improved direction would be wireless and small to easy to carry or even implanted in vivo. However, whether it is carrying or implanted (particular implanting) should cause interruption of treatment or research caused by replacing the battery. If the battery could be infinite charged, the best state for treatment or research can be achieved and without multiple injuries for the subjects. This new design of the stimulation mode is the most important application.

### 6. Inventive Motivation

In recent years, the digital diagnostic technique continues to make a breakthrough or innovation. The quantitative values of body heart rate, autonomic nervous activity and physical activity can be accurately obtain thorough using three-axis accelerative and electrocardiogram signals after processing digital signals. However, in medical research, these changes in physiological parameters are known to concern with physical health, disease prognosis and death forecast, so that it is proper to use as the applications for healthy elderly and patients monitoring and early warning. The measurement of physiological electrical signals now can be implemented by non-invasive and wireless transmission. As long as contacting with the skin, the physiological signals can wirelessly transmitted real-time. Further, a novel inductive charging concept can be added to enable ultra-long wear, and the application thereof could be broader and more appropriate. After simple designing, long time tracing can provide more complete care for the subjects (people and animals), besides monitoring, recording and correct analyzing

### SUMMARY OF THE INVENTION

Heart rate, activity signals are always an important indicator for signs of life or animal behavior. The collection and analysis for physiological signals will help the understanding of many health care information and future medical applications. Thus, normal physiological phenomenon could be responded by the design of wireless telemetry. In addition, monitoring more long-time (super long) change will understand various physiological functions more accurately and also provide medical information more effectively. Furthermore, proving continuing electrical stimulation in therapy or research will enable to achieve the ultimate long-term treatment. Recently, sensing and receiver techniques for various micro-sensor wireless signals have matured, various physiological signal analysis has progressed, and various induction charging designs have matured, and thus effective and reliable wireless monitoring and stimulating system can be achieved. The inventor has recently developed a lot of related hardware and software products and thus an efficient and reliable ultra-long time recording and stimulation system for people use and animal use would be provided by slightly designing and improving.

In order to achieve the mentioned inventive objects, the present invention provides a physiological signal sensing system without time and place constraint. The physiological signal sensing system includes an electrocardiogram collector, a neural activity analyzer, a wireless transceiver, a remote data processing device and a rechargeable battery. The electrocardiogram collector is used for detecting and recording electrocardiogram data. The electrocardiogram collector includes detecting electrode and a reference electrode to be mounted on the skin surface, thereby achieving the purpose of the non-invasive measurement. The neural activity analyzer is used for analyzing neural activity from electrocardiogram data collected by the electrocardiogram collector. For example, heartbeat cycle sequence can be obtained from electrocardiogram data collected by the electrocardiogram collector through a peak detection program other methods with the noise separation program. Then, the spectrum analysis can convert sampling results of the heartbeat cycle into a power density spectrum to calculate power of low-frequency (0.04˜0.15 Hz) and high-frequency (0.15˜0.4 Hz) from the power density spectrum through quantitative integration. These power and heartbeat cycle sequence can represent some neural activities. The wireless transceiver is used for transmitting the electrocardiogram signals collected by the electrocardiogram collector or the processed neural activity data to the remote data processing device, or receiving data from the remote data processing device. The remote data processing device is used for receiving and analyzing the obtained signals and returning results to the wireless transceiver or sending them to a medical organization. The rechargeable battery is used for maintenance operation of the electrocardiogram collector and the wireless transceiver, the rechargeable battery is charged by a contactless mode, thereby providing a continuous flow of power supply.

In brief, the present invention provides a physiological signal sensing system without time and place constraint, includes:

an electrocardiogram collector;

a neural activity analyzer, for analyzing neural activity from electrocardiogram data collected by the electrocardiogram collector;

a wireless transceiver; and

a remote data processing device;

wherein the wireless transceiver is used for transmitting electrocardiogram signal collected by the electrocardiogram collector or the processed neural activity data to the remote data processing device or receiving data from the remote data processing device, and the remote data processing device is used for receiving and analyzing the obtained signal and returning results to the wireless transceiver or sending them to a medical organization.

According to different medical or experimental purposes, the present system could includes a radio stimulating device or other feedback device for giving a user an electrical stimulation treatment, a medical suggestion or a warning.

In a preferred embodiment, the electrocardiogram collector includes a non-invasive measuring device for measuring the electrocardiogram data. The non-invasive measuring device includes a detecting electrode and a reference electrode, and the detecting electrode and a reference electrode are mounted on the skin surface.

In another preferred embodiment, the present system further includes a radio stimulating device to give a user an electrical stimulation treatment. Alternatively, the present system further includes feedback device for suggesting or warning the user.

In a preferred embodiment, the present system further includes battery for maintenance operation of the electrocardiogram collector and the wireless transceiver and charging with a contactless mode.

Since the body activity can reflect illness, health status and lifestyle, in particular, is used to assess the quality of sleep. Thus, in a preferred embodiment, the present system further includes:

a acceleration sensing component, collecting signals and signal processing results, and sending them to the remote data processing device via the wireless transceiver; and

a physical activity calculating device for calculating a physical activity from the signals collected by the acceleration sensing component.

The acceleration sensing component is used for measuring three-axis acceleration and calculating the body activity give corresponding suggestions and feedback.

In a preferred embodiment, the wireless transceiver and the remote data processing device respectively include a transmission interface which is a wireless communication protocol selected from a group of radio, wireless network, infrared, Bluetooth, radio frequency, GSM, PHS and CDMA. In a preferred embodiment, the contactless mode is a charging process selected from a group of induction, optoelectronics, piezoelectricity, acousto-electricity, wind power and thermal electricity.

In a preferred embodiment, the neural activity analyzer is used for carrying on spectral analysis to the heartbeat data.

The present invention also provides a physiological signal sensing method without time and place constraint, includes:

measurement of electrocardiogram signals;

wireless transmission of data; and

contactless charging of equipment.

In a preferred embodiment, the measurement of electrocardiogram signals is a non-invasive process. The wireless transmission is a wireless communication protocol selected from a group of radio, wireless network, infrared, Bluetooth, radio frequency, GSM, PHS and CDMA. The contactless charging is a charging process by one selected from a group of induction, optoelectronics, piezoelectricity, acousto-electricity, wind power and thermal electricity.

In another preferred embodiment, the present method further provides that the measured electrocardiogram signals would be implemented by analysis of physical activity, electrotherapy or feedback response in accordance with different purposes.

The details and the embodiments in the present invention are set forth in the following detailed description taken in conjunction with the accompanying drawings

### BRIEF DESCRIPTION OF THE DRAWINGS

FIG. 1 is a schematic view showing a block diagram of the physiological signal sensing system according to the embodiment of the present invention.

FIG. 2 is a schematic view showing an inductive charger circuit diagram.

### DETAILED DESCRIPTION OF THE PREFERRED EMBODIMENTS

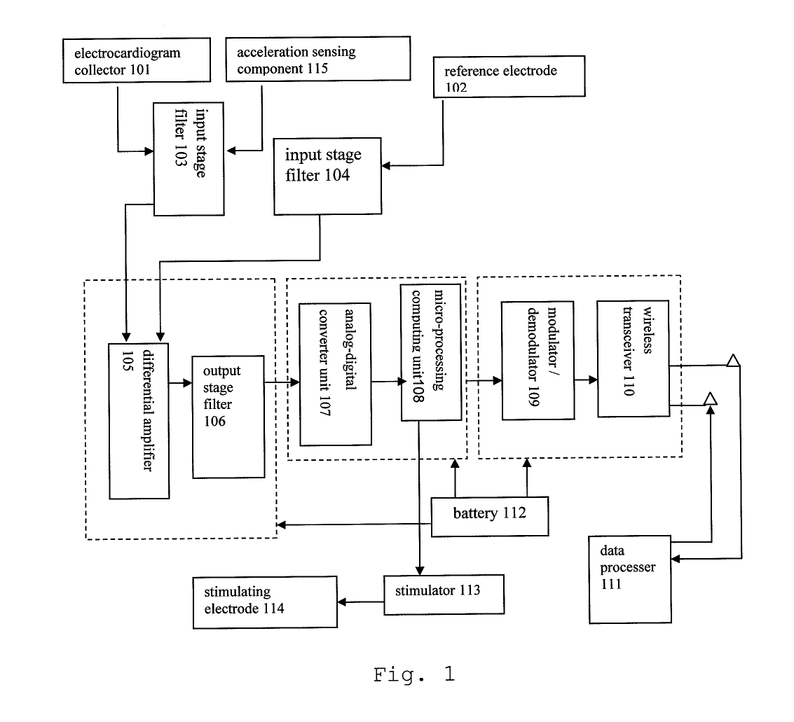

The present invention provides a physiological signal sensing system shown in FIG. 1. The system includes an electrocardiogram collector 101, a wireless transceiver 110, a battery 112, an acceleration sensing component 115, a reference electrode 102, input stage filters 103/104, a differential amplifier 105, an output stage filter, an analog-digital converter unit 107, a micro-processing computing unit 108, a modulator/demodulator 109, a stimulator 113 and a stimulating electrode 114 to integrate into a sensing stimulator. If the sensing stimulator is used in the human body, it can be designed to be the necklaces shape and be worn around the neck. Alternatively, if the sensing stimulator is used in the animal body, it would be implanted in the animal.

The electrodes in two ends of the electrocardiogram collector would form a basic circuit collected by potential signals. In order to simplify the use method and increase the reliability, the electrocardiogram collector uses two-electrode input method. However, the two-electrode input method includes more serious noise interference than the three-electrode differential input. This problem can be overcome by a filter circuit and an optical isolation circuit. For example, the present invention could use an amplifier circuit in the prior art (Kuo 1999) to amplify electrocardiogram input of the two electrodes thereby obtaining practical pattern of signal to noise ratio. The necklace-type electrocardiogram and body temperature signals may be intermittent because of user mobility. The user has a request for measuring, and the fixed electrode will stabilize the signal with at least 5 minutes. In addition, the noise can be processed by a specific process. For the animal use, it could be modified according to the same principle.

Digital electrocardiogram and pulse signals proceeds the following processes (Kuo et al. 1999; Yang et al. 2000): Through the peak detection program (Kuo and Chan 1993), the highest point of each heartbeat fluctuation is found out as the representative of each heartbeat. The computer program measures the height, duration and other parameters form the representative of each heartbeat, and the mean and standard deviation of the parameters will be calculated as a standard template. Next, each heartbeat is compared with the standard template. If the compared result of a heart rate is behind three standard deviations of the standard template, it would be considered to be the noise and will be deleted. Then, it is measured for the interval between two neighboring heartbeats peaks as the heartbeat of the cycle. The mean and standard deviation for all the heartbeat cycles are calculated to confirm all of the heartbeat cycles. If one heartbeat cycle is fell outside the three standard deviation, it should be considered to be noise or an unstable signal to delete it. The heart cycle sequence passed through this identification process will be analyzed.

All qualified heartbeat cycle sequences are sampled and retained with the frequency of 7.11 Hz to maintain continuity of their time. The spectrum analysis uses a Fourier method. First, a linear signal drift should be eliminated to prevent the interference of low-frequency band and to avoid the spectrum of individual frequency components of the mutual leakage used by hamming operations (Kuo 1999 et al.; Kuo and Chan 1993). Next, the power density spectrum would be obtained by taking 288 seconds data (2048 points) and using the fast Fourier transform within (Cooley and Turkey 1965). The effects caused by sampling and hamming operations are compensated (Kuo 1999; Kuo et al. 1999).

The power density spectrum of heart rate variability quantifies two frequency bands thereof through integral, and the two frequency bands include power of low-frequency (LF, 0.04-0.15 Hz) and high-frequency (HF, 0.15-0.4 Hz)). Further, total power (TP), LF/HF and other quantization parameters can be obtained (Anonymous 1996; Kuo et al. 1999; Yang et al. 2000). These parameters are converted by the logarithmic transformation to achieve an normal distribution (Kuo et al. 1999). Furthermore, the present invention for use in animals should adjust its frequency range based on different animals.

According to the previous experiment (Kuo et al. 1999; Kuo et al. 1997; Yang et al. 2000; Yien et al. 1997) and the consensus in Europe and the United States cardiologists (Anonymous 1996), the results are that HF in human or animal is an indicator of cardiac parasympathetic activity, LF/HF is an indicator of cardiac sympathetic nerve activity, and LF is an integration indicator of autonomic nervous activity.

The acceleration sensing component is a acceleration sensor IC (or collected by other methods), which can measure three-axis direction (x-axis, y-axis, z-axis) acceleration and obtain a total acceleration of \sqrt{x²+y²+z²} by using a program to consolidate three direction acceleration into a single signal as the subject's activity.

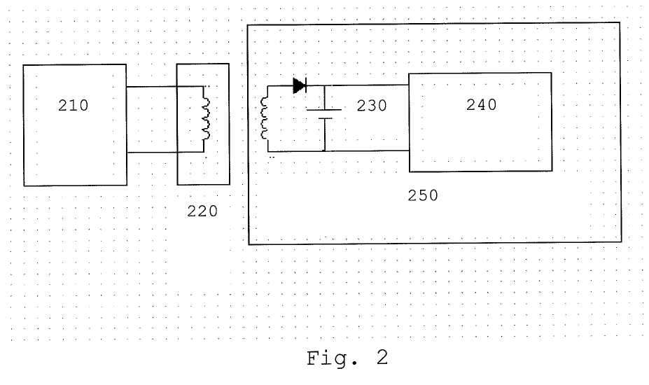

The radio stimulation can select a cardiac rhythm device, a nerve stimulator, a deep brain nucleus stimulator, a muscle stimulator, a gastrointestinal stimulator and so on according to the purpose. Power required by the electrical stimulator and other parts of the present system are provided by inductive rechargeable battery, and its circuit diagram is shown in FIG. 2. Inductive charging coil 220 and an inductive rechargeable wireless sensor 250 can are interacted by a high-frequency oscillating circuit 210, so that a battery 230 will be charged by a sensor circuit 240. The inductive charging device can be put in the place which can stay for a while every day, such as in bedside.

While the invention has been described in terms of what are presently considered to be the most practical and preferred embodiments, it is to be understood that the invention need not to be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A physiological signal sensing system without time and place constraint, comprising:
an electrocardiogram collector;
a neural activity analyzer, analyzing neural activity from electrocardiogram data collected by the electrocardiogram collector;
a wireless transceiver; and
a remote data processing device;
wherein the wireless transceiver is used for transmitting electrocardiogram signal collected by the electrocardiogram collector or the processed neural activity data to the remote data processing device or receiving data from the remote data processing device, and the remote data processing device is used for receiving and analyzing the obtained signal and returning results to the wireless transceiver or sending them to a medical organization.

2. The physiological signal sensing system of claim 1, wherein the electrocardiogram collector comprises a non-invasive measuring device for measuring the electrocardiogram data.

3. The physiological signal sensing system of claim 2, wherein the non-invasive measuring device comprises a detecting electrode and a reference electrode, and the detecting electrode and a reference electrode are mounted on the surface of the skin.

4. The physiological signal sensing system of claim 1 further comprising a radio stimulating device to give a user an electrical stimulation treatment.

5. The physiological signal sensing system of claim 1 further comprising a feedback device for suggesting or warning a user.

6. The physiological signal sensing system of claim 1, wherein the wireless transceiver and the remote data processing device respectively include a transmission interface which is a wireless communication protocol selected from a group of radio, wireless network, infrared, Bluetooth, radio frequency, GSM, PHS and CDMA.

7. The physiological signal sensing system of claim 1 further comprising a battery for maintenance operation of the electrocardiogram collector and the wireless transceiver and charging with a contactless mode

8. The physiological signal sensing system of claim 7, wherein the contactless mode is a charging process selected from a group of induction, optoelectronics, piezoelectricity, acousto-electricity, wind power and thermal electricity.

9. The physiological signal sensing system of claim 1 further comprising:
a acceleration sensing component, collecting signals and signal processing results and sending them to the remote data processing device via the wireless transceiver;
a physical activity calculating device for calculating a physical activity from the signal collected by the acceleration sensing component.

10. The physiological signal sensing system of claim 1, wherein the neural activity analyzer is used for carrying on spectral analysis to the heartbeat data.

11. A physiological signal recording method without time and place constraint, comprising:
measurement of electrocardiogram signals;
wireless transmission of data; and
contactless charging of equipment.

12. The physiological signal recording method of claim 11, wherein the measurement of electrocardiogram signals is a non-invasive process.

13. The physiological signal recording method of claim 11, wherein the contactless charging is a charging process by one selected from a group of induction, optoelectronics, piezoelectricity, acousto-electricity, wind power and thermal electricity.

14. The physiological signal recording method of claim 11, wherein the measured electrocardiogram data is proceeded through a nervous activity analysis.

15. The physiological signal recording method of claim 11, wherein the wireless transmission is a wireless communication protocol selected from a group of radio, wireless network, infrared, Bluetooth, radio frequency, GSM, PHS and CDMA.

16. The physiological signal recording method of claim 11 further comprising analysis of physical activity.

17. The physiological signal recording method of claim 11 further comprising electrotherapy.

18. The physiological signal recording method of claim 11 further comprising a feedback response
